(19) United States
(12) Patent Application Publication
(10) Pub. No.: US 2016/0256472 A1
(43) Pub. Date: Sep. 8, 2016

(54) COMPOSITIONS AND METHODS OF ADMINISTERING SAME

(71) Applicant: PALMAYA PTY LTD, Western Australia (AU)

(72) Inventor: Alexander George Brian O'Neil, Subiaco WA (AU)

(21) Appl. No.: 15/028,146

(22) PCT Filed: Oct. 14, 2014
(86) PCT No.: PCT/AU2014/000978

§ 371 Date: Apr. 8, 2016

(30) Foreign Application Priority Data
Oct. 14, 2013 (AU) 2013903955

(51) Int. Cl. A61K 31/5517; A61K 31/138; A61K 31/4168; A61K 31/485

(52) U.S. Cl. CPC A61K 31/5517; A61K 31/485; A61K 31/138; A61K 31/4168

(57) ABSTRACT

A composition comprising two or more active agents in a micro-dose amount, such as a micro-dose of naltrexone in combination with a micro-dose of flumazenil, in the treatment of craving or anxiety resulting in rapid settling of the craving for substance use, smoking or an obsessional behaviour, preferably within minutes of administration of the composition.

### TECHNICAL FIELD

A composition comprising a micro-dose of naltrexone in combination with a micro-dose of flumazenil and methods of administering same for the treatment of cravings and anxiety. Other agents may also be combined with the naltrexone/flumazenil combination for the treatment of cravings and anxiety.

### BACKGROUND ART

The following discussion of the background art is intended to facilitate an understanding of the present invention only. The discussion is not an acknowledgement or admission that any of the material referred to is or was part of the common general knowledge as at the priority date of the application.

Opioid dependence is an addiction to opioids, such as drugs used to treat pain or heroin. The opioids act on the opioid receptors and an individual may develop an addiction to the opioids. Craving for the opioid is associated with opioid dependence. In addition, alcohol and non-opiate drugs such as nicotine, alcohol, marijuana, benzodiazepines, amphetamines, gambling and meditation all produce a feeling of pleasure through elevations of dopamine and opiates by the endogenous release of these substances. These effects may be reduced by the presence of naltrexone having a blocking effect at the opioid receptor sites and so reduce pleasure from the substance or behaviour and also reduces craving for the substance or behaviour.

Naltrexone is a drug most commonly used in oral form to assist in the detoxification or long term maintenance of opioid dependence, and also with alcohol addiction. Naltrexone blocks the effects of opioids by blocking the opioid receptor. It is thought that if a person does not experience any positive effect on repeated exposure when using the opioid, that person usually will eventually cease opioid or alcohol use. The literature shows that naltrexone has a limited but useful place in treating addiction but by itself has a high failure rate with managing addiction outside opiates or alcohol. A recent Cochrane review on smoking confirms that it is not effective with smoking addiction.

Flumazenil is an antagonist at the benzodiazepine section of the GABA receptor. It has mainly been used commercially in the management of benzodiazepine overdose where it returns patients to consciousness and returns the respiratory drive back to normal. It has been available for 30 years and is only commercially used intravenously with a purpose to resuscitate benzodiazepine overdose patients. The literature describes the fact that intravenous flumazenil may block the benzodiazepine receptor sites and is associated with side effects such as panic attacks or fitting.

The literature on flumazenil suggests it may have a role in the treatment of a number of addictions. Clinically, however there is no well-established place for flumazenil in addiction management. Furthermore, there appears to be no significant literature on the use of flumazenil in the treatment of addiction to smoking cigarettes or marijuana.

Bupropion is an antidepressant or anti-smoking agent. Although the exact mechanism of action of bupropion is unknown, it has been suggested to be an inhibitor of norepinephrine-dopamine reuptake. In use, bupropion is prescribed as an oral formulation. For example, in immediate release formulations, bupropion is administered at 100 mg per dose, twice a day. The dose may be increased to as much as 300 mg per day (3×100 mg tablets) as a maintenance dose, and up to 450 mg per day. A sustained release tablet is also available, which is administered as a 150 mg oral dose once a day. This dose may be increased up to a maximum of 200 mg twice per day. Extended release dosage forms are also available, reaching an oral dose of up to 450 mg per day.

Clonidine is used in the treatment of hypertension. However, it may also be prescribed to treat Attention Deficit Disorder (ADD), Attention Deficit Hyperactivity Disorder (ADHD), anxiety, migraines, withdrawal symptoms and pain conditions. Clonidine is usually administered as an oral (tablet) dosage form. Depending on the disease/disorder to be treated, the dosage varies from 200 micrograms per day to up to 600 micrograms per day. Patches may also be used to administer the drug, usually at a rate of 100 micrograms per 24 hours. Clonidine is an imidazoline receptor antagonist.

As with all addictions, craving anxiety builds and is then reduced at the time of drug use or when the habit is repeated. The cycle then repeats itself. Craving may present itself with anxiety or in some cases presents itself where the individual places himself in a risky situation or searches for the substance or opportunity for the substance or event. With oral naltrexone the absorption from the stomach is slow (up to a hour with the added problem of liver breakdown of naltrexone) and so the time waiting for correction of craving following an oral dose of naltrexone is so long that the craving will have often driven the individual to use before the usual oral dose has had any effect on craving for alcohol, smoking or drug use.

Even with the existing treatments, dependence is still a major problem. Therefore, there exists a need to develop a composition and methods of administering same for the treatment of severe craving which will cause rapid settling of the craving for substance use, smoking or an obsessional behaviour, preferably within minutes of administration of the composition.

### SUMMARY OF INVENTION

Applicant has surprisingly found that it is possible to treat cravings by the use of micro-doses of naltrexone in combination with micro-doses of flumazenil, delivered either by rapid delivery or bolus delivery including intravenous, trans-pulmonary, nasal, sublingual and rectal doses which results in a sudden drop in craving induced anxiety within 15 minutes for most patients. It has been found that the synergistic effect of both drugs together given as repeated bolus doses each time a craving for the drug use or behaviour builds results in a reduction in craving and anxiety to a low or controlled level. This is especially surprising with a drug like flumazenil, which has a history of causing panic attacks.

It has also be found that it is possible to treat cravings by the use of micro-doses of other opioid antagonists such as naloxone and nalmephene in combination with micro-doses of flumazenil, delivered either by rapid delivery or bolus delivery including intravenous, trans-pulmonary, nasal, sublingual and rectal doses which results in a sudden drop in craving induced anxiety within 15 minutes for most patients.

Furthermore, it has been found that it is also possible to treat cravings by the use of micro-doses of bupropion in combination with micro-doses of flumazenil and micro-doses of naltrexone (or naloxone or nalmephene), delivered either by rapid delivery or bolus delivery including intravenous, trans-pulmonary, nasal, sublingual and rectal doses which results in a sudden drop in craving induced anxiety within 15 minutes for most patients.

The applicant has also found that is possible to treat cravings by the use of micro-doses of clonidine in combination with micro-doses of flumazenil and micro-doses of naltrexone (or naloxone or nalmephene), delivered either by rapid delivery or bolus delivery including intravenous, trans-pulmonary, nasal, sublingual and rectal doses which results in a sudden drop in craving induced anxiety within 15 minutes for most patients.

In one aspect of the present invention there is provided a composition comprising a micro-dose of naltrexone and a micro-dose of flumazenil.

In one embodiment of the present invention, the naltrexone can be replaced with either naloxone or nalmephene at the same micro-dose amount.

In a preferred embodiment, the micro-dose of naltrexone (or naloxone or nalmephene) or flumazenil ranges from about 10 microgram to about 2000 micrograms. In a highly preferred embodiment, the micro-dose of naltrexone or flumazenil is 250 micrograms.

In a further aspect of the invention there is provided a composition comprising naltrexone to flumazenil in a ratio by weight of from 0.1:10, wherein the minimum does of naltrexone is 10 micrograms and the maximum dose of flumazenil is 2000 micrograms.

In a further aspect of the invention there is provided a composition comprising flumazenil to naltrexone in a ratio by weight of 0.1:10, wherein the minimum dose of flumazenil is 10 micrograms and the maximum dose of naltrexone is 2000 micrograms.

In a highly preferred embodiment of the present invention, naltrexone to flumazenil is in a ratio of 1:1.

As discussed above, naltrexone may be replaced with any one of naloxone or nalmephene.

In a further aspect of the present invention there is provided a composition comprising micro-doses of flumazenil and naltrexone in a ratio wherein the flumazenil and naltrexone (or naloxone or nalmephene) are absorbed rapidly so as to result in the treatment of craving or anxiety within a period of time of less than 20 minutes.

In a preferred embodiment, the composition is administered so as to achieve rapid naltrexone (or naloxone or nalmephene) and flumazenil delivery or bolus delivery via trans-pulmonary, intravenous, nasal, rectal or sublingual delivery systems.

In a further aspect of the invention there is provided a use of naltrexone (or naloxone or nalmephene) and flumazenil in the preparation of a medicament for the treatment of craving or anxiety, or disorder or condition associated with craving or anxiety. There is also provided a use of bupropion and flumazenil and naltrexone in the preparation of a medicament for the treatment of craving or anxiety, or disorder or condition associated with craving or anxiety. Furthermore, there is also provided a use of clonidine and flumazenil and naltrexone in the preparation of a medicament for the treatment of craving or anxiety, or disorder or condition associated with craving or anxiety.

In another aspect of the present invention there is provided a composition comprising a micro-dose of bupropion, a micro-dose of naltrexone and a micro-dose of flumazenil. There is also provided a composition comprising a micro-dose of clonidine, a micro-dose of naltrexone and a micro-dose of flumazenil.

In a preferred embodiment, the micro-dose of bupropion, naltrexone (or naloxone or nalmephene) or flumazenil ranges from about 10 microgram to about 2000 micrograms. Preferably, the ratio of bupropion/naltrexone/flumazenil is 1:1:1. In a highly preferred embodiment, the micro-dose of bupropion, naltrexone or flumazenil is 250 micrograms each.

In a further aspect, the present invention provides a composition a micro-dose of clonidine, a micro-dose of naltrexone and a micro-dose of flumazenil.

The micro-dose of clonidine in the composition ranges from 10 micrograms to 150 micrograms, whereas the micro-dose of naltrexone (or naloxone or nalmephene) or flumazenil ranges from about 10 microgram to about 2000 micrograms.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### DESCRIPTION OF EMBODIMENTS

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, displacement and field strength etc.). A range of values will be understood to include all values within the range, including the values defining the range, and values adjacent to the range which lead to the same or substantially the same outcome as the values immediately adjacent to that value which defines the boundary to the range.

The term neurological “disorders”, diseases”, or “conditions” may be used interchangeably to define a disease, disorder or condition which effects the body's nervous system.

The term “dependence” may apply to opioids, alcohol, smoking nicotine, marijuana, cocaine, amphetamines, other substances, obsessional behaviour characterised by gambling, temper tantrums, or episodes of terror or post-traumatic stress disorders.

The term “craving” is defined as an increasing desire or need that gradually controls the individual's behaviour until the desired substance or experience is achieved.

The term “active agent” may mean one active agent, or many encompassing two or more active agents.

The term “micro-dose” refers to a lower than expected dose of the active agents naltrexone (or naloxone or nalmephene), flumazenil, bupropion or clonidine for use in the treatment of individuals in need thereof. Therefore a micro-dose refers to an amount of from about 10 micrograms to about 2000 micrograms of naltrexone or flumazenil or bupropion, or about 10 micrograms to about 150 micrograms of clonidine.

The term “dependency” generally refers to a craving for, habituation to, or addiction to a chemical or other substance.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

Throughout this specification, unless the context requires otherwise, the word “comprise” or variations such as “comprises” or “comprising”, will be understood to imply the inclusion of a stated integer or group of integers but not the exclusion of any other integer or group of integers.

### DETAILED DESCRIPTION OF THE INVENTION

The composition of the present invention comprises a micro-dose of naltrexone in combination with a micro-dose of flumazenil. The composition is useful in the treatment of cravings and anxiety associated with severe craving resulting in the rapid settling of the craving for substance use, smoking or an obsessional behaviour, preferably within minutes of administration of the composition.

This invention relates to the fact that endogenous opioids are thought to be increased as one meditates on drug use before the use starts and endogenous opioids and dopamine increase in response to substances and events such as marijuana, opioids, alcohol, amphetamines, benzodiazepines, other drugs, gambling, obsessional or risky behaviour. The use of small bolus doses of naltrexone and flumazenil are suggested to serve to reduce the tolerance that develops when high levels of endogenous opioids or opioids are given to an individual.

The bolus dose of naltrexone and flumazenil is suggested to correct acute tolerance before or after drug use. Tolerance to endogenous opioids may occur with prolonged severe craving giving many pulses of endogenous opioids. This tolerance may mean that pulses of endogenous opioids no longer register after prolonged craving. By delivering, for example, a nasal spray of naltrexone, the acute tolerance caused by craving alone may be achieved and correct the receptor damage before drug use starts.

Without being bound by theory, it is suggested that tolerance develops in seconds. For example, smokers observe that the first puff of a cigarette is better than later puffs. Individuals that drink alcohol observe that the first glass of alcohol is better than later glasses. With a blood level of 0.8 there is an associate rise in “feeling terrific” and when the blood level is falling to 0.8 the anxiety with detoxing means that 0.8 is not as enjoyable. Hangovers after drinking binges are related to the drop in tolerance and the detoxing process causes anxiety and discomfort.

The change in tolerance with a rise in blood level of endogenous or exogenous opioids, and the anxiety sensitisation associated with this change in tolerance, all happen within minutes of each episode of drug or alcohol use. The concept of rapid delivery of naltrexone (or naloxone or nalmephene) (antagonists to opioids) and flumazenil (an antagonist to prevent anxiety and anxiety sensitisation) is a new innovative approach to treat increased tolerance and addiction at the moment it occurs in the individual.

When the compositions of the present invention are administered by nasal delivery, it is possible to administer the active agents in an amount hundreds of times below the toxic doses for these active agents to control symptoms. Typically, treatment is on an hourly dosage. The sudden rise in the levels of the active agent in the blood of the patient appears to give important symptom control—just when the blood level is rising and a short time after treatment. It appears the combination of two or more active agents as defined by the invention, and the rapid rise in the concentration of the active agents in the blood of the treated patient is critical for the efficacy of the treatment. In use, the compositions of the present invention may be administered 20 to 50 times a day for symptom control, without reaching toxic levels of the active agent in the treated patient.

Applicant has found, for example, that the sublingual or nasal delivery of 250 micrograms of naltrexone with 250 micrograms of flumazenil has a profound effect on craving and drug use. These methods of administration allow for a fast rate of delivery of the antagonists to the individual to treat the craving at the moment it occurs in the individual.

In clinical use we have found that these two antagonists delivered together gives symptom relief to patients often described by patients as ‘chilling out’ (reducing their craving and craving anxiety) just when they would have reached for a cigarette, drug or alcohol. In the time before drug or alcohol use the meditation on using is associated with endogenous opioid release in the nucleus accumbins. This opioid release and meditation occurs as a series of cycles and anxiety mediated by GABA neurotransmitters gradually rises until drug seeking activity occurs and a decision to use drugs occurs. In our clinical studies the use of naltrexone (250 micrograms) and flumazenil (250 micrograms) delivered sublingually or nasally allows the build-up of craving and craving related anxiety to settle. This combination of these two antagonists has not been described before. Furthermore, this pulsatile delivery of naltrexone and flumazenil by nasal or sublingual delivery has not been described for the control of addiction or its symptoms before.

The treatment regime of the present invention involves the delivery of micro-doses of naltrexone in combination with micro-doses of flumazenil, through rapid absorption methods, including but not limited to intravenous, nasal, sublingual or rectal delivery systems. It is expected that as the patient suffers a craving during the course of a 24 hour period, the patient will be exposed to a particular cue specific to the individual, and self-administer a dose of the composition of the present invention, with the resulting action that is sufficiently quick to reduce the craving quickly enough to redirect the individual's activity which would have otherwise led to deliver the substance or experience, within a short period of time.

This approach of providing a composition which will allow the rapid and repeated delivery of the composition which is an antagonist to control craving symptoms has not been described before. The rapid delivery of antagonists to the opiate and benzodiazepine receptor systems to control craving so as to achieve the synergistic effects of reduced craving through the opiate system and reduced anxiety through the GABA system has not been described before.

The concept of combining naltrexone (an opioid antagonist) and flumazenil (a benzodiazepine antagonist) is new. Both compounds are antagonists and so the development of tolerance seen with agonists is not expected. Furthermore, the concept of delivering naltrexone sublingually is new and has not been seen before. The fact that oral naltrexone disappears from the blood stream six hours after administering the oral dosage form means naltrexone is undetectable in the blood stream for 18 hours a day, using standard administration and in accordance with recommended dosage regimes, which is a one off dose of 50 mg per day.

The methods and compositions of the present invention teaches that as soon as craving occurs more naltrexone should be administered and in this way serum naltrexone levels can be achieved throughout a 24 hour period with multiple administrations of micro-doses of a combination of flumazenil and naltrexone. In the case of flumazenil, the half-life is less than one hour and therefore, multiple administrations of micro-doses without an intravenous or subcutaneous line is a practical administration method. The sublingual and nasal delivery systems allow repeat doses with a minimum of effort.

The present invention therefore is directed towards the reduction of craving using naltrexone and reducing craving anxiety using flumazenil and naltrexone delivered simultaneously.

Micro bolus doses of naltrexone in combination with micro bolus doses of flumazenil may be administered by any rapid delivery means known in the art. For example, the composition of the present invention may be administered via the following routes: intravenous, trans-pulmonary, sublingual, nasal or rectal delivery. Use in this way to control episodes of craving or anxiety has not been previously described.

It is known that craving generated anxiety eventually drives the individual to search for rewards or substances. Without being bound by theory, it is suggested that modifying the GABA system reduces craving induced anxiety. Investigations into the delivery of flumazenil to reduce craving for benzodiazepine addictions has been carried out. It has been shown from these studies that flumazenil modifies responses at the benzodiazepine receptor and the changes may assist with alcohol addiction in the same way that they do with benzodiazepine addiction.

Both benzodiazepine addiction and alcohol addiction involve responses at the GABA_{A} receptor and as such, flumazenil may directly modify GABA_{A} receptor responses to alcohol. Craving also has an effect at the GABA_{A} benzodiazepine receptor where an increase in inverse agonists may occur as craving induced anxiety increases. It is suggested that flumazenil directly block the effects of these inverse agonists or alternatively modify sensitivity to agonists at the benzodiazepine receptor.

The compositions of the present invention have at least the following advantage over the known treatments for dependence. That is, there is a greater than expected reduction of craving for a drug. In addition, there is evidence of an associated drop in sympathetic tone, drop in pulse rate and cessation of smoking, alcohol or other drug use. There has also been a reduction in the level of anxiety experienced by patients receiving the composition of the present invention.

The synergistic effects of the medicament of the present invention has also been associated with returning sleeping to normal patterns in disturbed sleep patients, and patients suffering from Parkinson disease.

The synergistic effects of the medicament of the present invention have been achieved by the method of administering a patient with a patient controlled dose of the medicament of the present invention at intervals as short as 10 minutes or less. If the bolus did not achieve a reduction in anxiety or craving anxiety, the composition of the present invention may be administered on demand until anxiety and or craving settles sufficiently.

The compositions of the present invention are capable of treating craving associated with all dependency conditions including opiates, smoking cigarettes, marijuana, cocaine, benzodiazepines, amphetamines, alcohol, and other substances including solvents and hallucinogenic substances.

Acute rapid reduction of craving and anxiety will also be useful in addictive behavioural and anxiety conditions such as: post-traumatic stress disorders, sleep disorders, all withdrawal situations, obsessive compulsive disorders, agitated psychiatric states, patients with psychosis and agitation from many different conditions. Thus, the compositions defined by the invention are suitable for the treatment to reduce or prevent the craving associated with opiates, smoking cigarettes, marijuana, cocaine, benzodiazepines, amphetamines, alcohol, and other substances including solvents and hallucinogenic substances.

The composition and methods of the invention are of value when craving and or anxiety causes difficulty in sleeping (e.g. insomnia). Thus, the compositions and methods of delivering the same provide a sleeping tablet that should not lead to tolerance problems which have been the key problem with other sleeping tablets.

The composition of the present invention comprises naltrexone and flumazenil.

It has further been found that the addition of bupropion in a micro-dose amount to the combination of flumazenil and naltrexone assists with the treatment of cravings and addictive behavioural and anxiety conditions in patients.

It has further been found that the addition of clonidine in a micro-dose amount to the combination of flumazenil and naltrexone assists with the treatment of cravings and addictive behavioural and anxiety conditions in patients.

### Naltrexone

The amount of naltrexone present in the composition of the present invention is less than the amount which is required in the absence of flumazenil.

Naltrexone was initially tested in combination with flumazenil. It has been found that other opioid antagonists such as naloxone and nalmephene may be used in place of naltrexone with similar results. Although naltrexone has been referred to below, it should be understood that naloxone or nalmephene may be used in place of naltrexone in the same micro-dose amounts.

Naltrexone is an antagonist used for alcohol addiction and is sufficiently lipophilic to be absorbed by sublingual tissues. The recommended oral daily dose of naltrexone in the treatment of alcohol addiction is 50 mg. Applicant has surprisingly found that a dose of at least 200 times less than recommended for the treatment of alcohol addiction is capable of treating opioid dependence. For example, the recommended daily dose for alcohol treatment in a patient is 50 mg per day, whereas the medicament of the present invention can be used in a dose of 250 micrograms per “craving” episode—which is 200 times less than the recommended daily dose.

The patient would have to take 200 doses per day to reach the same dose normally prescribed for treatment of an addiction. Therefore, the patient is provided with a safe dose to medication whenever the craving is considered severe. It is anticipated that most patients will not need more than 20 doses per day. It is expected that the rapid rise in naltrexone levels following the naltrexone dose will directly decrease opiate induced craving while the naltrexone levels are rising. By this mechanism craving for substances and other behaviours will be temporarily decreased.

Preferably, the compositions of the present invention comprise about 10 micrograms to about 2000 micrograms of naltrexone per unit dose. In a preferred embodiment, the composition comprises, at least 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1500, or 2000 micrograms of naltrexone per unit dose. In a highly preferred embodiment, the composition comprises 250 micrograms of naltrexone per unit dose.

### Flumazenil

The amount of flumazenil present in the composition of the present invention is less than the amount which is required in the absence of naltrexone.

Flumazenil was the first benzodiazepine receptor antagonist tested. However, it is anticipated that micro-doses of other benzodiazepine receptor antagonist may also achieve similar results in patients. For example, flumazenil as discussed herein may be replaced with any one of the following benzodiazepine receptor antagonists—alpha5 inverse agonist, pentlenetetrazole, bilobalide, or picrotoxin.

The dose for the composition of the present invention preferably uses 250 micrograms per dose of flumazenil. This contrasts with the 12 mg dose of flumazenil used by researchers investigating the sublingual use of flumazenil in the treatment of hypersomnia patients. Mims records the safety of doses of up to 100 mg of flumazenil administered via intravenous injection. The dose of the present composition is therefore a micro-dose of at least 48 times less than the sublingual doses used in hypersomnia patient study.

Preferably, the compositions of the present invention comprise about 10 micrograms to about 2000 micrograms of flumazenil per unit dose. In a preferred embodiment, the composition comprises, at least 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1500, or 2000 micrograms of flumazenil per unit dose. In a highly preferred embodiment, the composition comprises 250 micrograms of flumazenil per unit dose.

### Bupropion

The amount of bupropion present in the composition of the present invention is less than the amount which is required in the absence of naltrexone and flumazenil.

The dose for the composition of the present invention preferably uses 250 micrograms per dose of bupropion. This contrasts with the 100 mg to 450 mg per day dose of bupropion used in the treatment of major depressive disorder or cessation of smoking in patients.

Preferably, the compositions of the present invention comprise about 10 micrograms to about 2000 micrograms of bupropion per unit dose. In a preferred embodiment, the composition comprises, at least 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1500, or 2000 micrograms of bupropion per unit dose. In a highly preferred embodiment, the composition comprises 250 micrograms of bupropion per unit dose.

### Clonidine

The amount of clonidine present in the composition of the present invention is less than the amount which is required in the absence of naltrexone and flumazenil. Clonidine may be administered in combination with naltrexone and flumazenil composition.

The dose for the composition of the present invention preferably uses 15 micrograms per dose of clonidine. This contrasts with the 200 micrograms to 600 micrograms per day dose of clonidine currently used in the treatment of hypertension, ADD, ADHD, anxiety, withdrawal symptoms and pain control in patients.

Preferably, the compositions of the present invention comprise about 10 micrograms to about 150 micrograms of bupropion per unit dose. In a preferred embodiment, the composition comprises, at least 10, 11, 12, 13, 14, 15, 16, 17, 18, 19, 20, 25, 30, 40, 50, 60, 70, 80, 90, 100, 110, 120, 130, 140, or 150, micrograms of clonidine per unit dose. In a highly preferred embodiment, the composition comprises 15 micrograms of clonidine per unit dose.

### Formulations of the Composition

The compositions of the present invention may be formulated in a number of different forms for the rapid delivery of naltrexone (or naloxone or nalmephene) and flumazenil, or bupropion/naltrexone/flumazenil or clonidine/naltrexone/flumazenil via trans-pulmonary, intravenous, nasal, sublingual and rectal bolus dose. That is, the composition of the present invention may be prepared by any means that allows rapid delivery of the mixture, which bypasses the stomach and so bypasses the liver, which breaks down the two antagonists, naltrexone and flumazenil, and bupropion.

Compositions of the invention may be delivered in the form of transdermal and trans-mucosal administration. Trans-mucosal delivery includes the delivery of the composition of the invention through the mucosa, including rectal mucosa, nasal mucosa and oral routes.

Compositions of the invention may be in the form of sublingual preparations, such as lozenges and tablets that are suitable for sublingual administration. The lozenges and tablets may contain soluble excipients selected from materials such as lactose, mannitol, dextrose, sucrose or mixtures thereof. They may also contain granulating and disintegrating agents, such as starch, binding agents and lubricating agents or flavourings.

Examples of sublingual preparations include naltrexone and flumazenil power mixed in a flavoured wax preparation which dissolves in the mouth. Wafers or rapidly dissolving tablets are an alternative. Bupropion or clonidine may also be added to this mixture.

Nasal spray preparations are rapidly absorbed. A person skilled in the art would be familiar with the types of nasal spray devices for use in delivering the composition of the invention. In a preferred embodiment, the individual dose of flumazenil and naltrexone per delivery is between about 10 μg to 2000 μg of naltrexone and 10 μg to 2000 μg flumazenil per does. In a highly preferred embodiment the amount of naltrexone delivered per dose is 250 μg and the amount of flumazenil delivered per dose is 250 μg.

In the embodiment where the composition comprises bupropion/flumazenil/naltrexone, the individual dose of bupropion, flumazenil and naltrexone per delivery is between about 10 μg to 2000 μg bupropion, 10 μg to 2000 μg of naltrexone and 10 μg to 2000 μg flumazenil per dose. In a highly preferred embodiment the amount of bupropion delivered per dose is 250 μg, naltrexone delivered per dose is 250 μg and the amount of flumazenil delivered per dose is 250 μg.

When the composition of the present invention comprises clonidine, the individual dose of clonidine, flumazenil and naltrexone per delivery is between about 10 μg to 2000 μg clonidine, 10 μg to 2000 μg of naltrexone and 10 μg to 2000 μg flumazenil per dose. In a highly preferred embodiment the amount of clonidine delivered per dose is 15 μg, naltrexone delivered per dose is 250 μg and the amount of flumazenil delivered per dose is 250 μg.

Subcutaneous or intravenous lines that allow the delivery of a bolus dose of the compositions of the present invention also will allow rapid bolus deliver of compositions comprising (i) naltrexone and flumazenil; (ii) bupropion, naltrexone and flumazenil; or (iii) clonidine, naltrexone and flumazenil. Therefore, rectal delivery of the compositions of the present invention may be practical. In one embodiment of the present invention the bolus dose of naltrexone in combination with flumazenil is in a suppository preparation. In another embodiment the present invention provides a bolus dose of bupropion, naltrexone and flumazenil; or a bolus dose of clonidine, naltrexone and flumazenil in a suppository preparation.

The methods and techniques of manufacturing suitable compositions for delivery to an individual in need thereof are known to the person skilled in the art. The compositions of the invention and the methods of delivery thereof allow rapid delivery of (i) both naltrexone and flumazenil; (ii) bupropion, naltrexone and flumazenil; or (iii) clonidine, naltrexone and flumazenil so as to gain control of symptoms when craving levels are high.

### Craving and Anxiety

The compositions and methods of administering same are useful in the treatment of craving and anxiety. The craving and anxiety may result from addictive behavioural and anxiety conditions such as: post-traumatic stress disorders, sleep disorders, all withdrawal situations, obsessive compulsive disorders, agitated psychiatric states, patients with psychosis and agitation from many different conditions. Thus, the compositions defined by the invention are suitable for the treatment to reduce or prevent the craving associated with, but not limited to: opiates, smoking cigarettes, marijuana, cocaine, benzodiazepines, amphetamines, alcohol, and other substances including solvents and hallucinogenic substances.

### EXAMPLES

Examples 1 to 4 and 7 to 9 describe results from pilot studies with the medicaments of the present invention. Examples 5 and 6 describe a study design for evaluating naltrexone and flumazenil given in micro-doses to control craving and anxiety symptoms for alcohol and other drug use.

### Example 1

A 62 year old woman presented at the age of 56 with a seven (7) year history of smoking 20 cigarettes a day and had no success with previous attempts at smoking cessation.

The woman was administered a composition comprising 50 microgram of naltrexone and 50 microgram of flumazenil via a nasal spray. She observed a decrease in craving and craving anxiety for smoking within 5 minutes of receiving the composition of naltrexone and flumazenil. About 40-50 minutes later the craving anxiety for smoking started to climb. She continued to give further doses of the composition each time the craving rose for a 6 week period and has not had another cigarette for greater than 5 years.

### Example 2

A 55 year old man smoking 35 to 50 cigarettes per day attempted to give up cigarettes trying all of the available pharmacotherapies without success. He reported that nasal delivery of naltrexone (50 micrograms) and flumazenil (50 micrograms) gave a profound reduction of smoking craving and anxiety. This allowed him to cease smoking provided he continued with the treatment. Cessation of the treatment caused him to relapse. Given that the adhoc dosing did not result in the cessation of smoking, he was then treated with naltrexone implants delivering and estimated 15 mg per day of naltrexone and flumazenil implants delivering an estimated 1-3 mg per day. He has now ceased smoking cigarettes for 4 months.

### Example 3

A 42 year old male gave a history of smoking and alcohol use from the age of 13 to 42. His craving for smoking and alcohol were linked making smoking more severe during times of alcohol use. He reported no improvement on any of the smoking pharmacotherapies available in Australia.

He was given a lozenge containing 250 micrograms of naltrexone and 250 micrograms of flumazenil to suck on until the lozenge dissolved in his mouth. He reported a profound reduction in craving anxiety and repeated the dose every time the craving anxiety returned. Fourteen (14) days later he had not smoked or returned to alcohol use. This profound change depends on him giving himself one or two doses before getting out of bed in the morning as this is the most severe craving in the day.

This patient also reported that a dose of 4 lozenges was effective as a sleeping tablet and allowed a return to a normal sleeping pattern that had previously been impossible to him. He also reported that his anxiety problems which affected his daily routines had resolved. He averaged 20 doses per day.

### Example 4

A 23 year old neuroscience student smoking 15 cigarettes a day presented and was given sublingual lozenges with 250 micrograms of flumazenil and 250 micrograms of naltrexone together. She also had a drinking problem consuming about nine (9) standard drinks per drinking episode.

After administration of the lozenges, she reported a profound reduction in craving anxiety within 10 to 15 minutes of each dose. This was associated with a drop in pulse rate by an average of 10 beats per minute and profound relaxation. Although she did drink alcohol on the third day and did not use cigarettes while drinking, the drinking episode was extremely unpleasant to the level she committed to avoid drinking while on this medication. Nine (9) days after starting she has not returned to smoking or drinking and needs about 10-15 doses per day for craving anxiety.

These four case reports lead us to the conclusion that they might represent many hundreds of craving episodes of craving anxiety where each episode of craving anxiety responded within 10-15 minutes of the naltrexone/flumazenil mixture delivered by the nasal and sublingual routes.

The ideal dose may vary from below 50 micrograms to up to 12 mg but the combination appears to be profoundly effective. Our observations are extremely surprising when contrasted with the most recent Cochran review on naltrexone and smoking which states that naltrexone research does not give any evidence of smoking cessation.

### Example 5

### A Randomised Controlled Trial Evaluating Naltrexone and Flumazenil Given in Micro-Doses to Control Craving and Anxiety Symptoms for Alcohol and Other Drug Use

This study is focused on assisting young people under the age of 24 with a new tool to decrease their craving for alcohol, cigarettes, marijuana and other drugs. Thus the study is aimed at determining whether the proposed medication is able to reduce the craving for drug use and thus reduce the use of alcohol, cigarettes, marijuana and other drugs. This study is also aimed at the evaluation of whether any benefits achieved by the medication are able to be sustained over a 3-month period.

There is a large body of literature which suggests that alcohol and smoking both increase the level of opiates in the body, and that use of alcohol and smoking in particular, is closely linked. Most of the publications reviewed by the authors suggest that smoking and alcohol usage were linked in up to 95% of patients.

In the past, patients have received small doses of naltrexone and flumazenil to control symptoms but this has been based on symptom-control in selected patients. These pilot studies are continuing to define the value of using a medication that will decrease craving and anxiety as anxiety build up prior to smoking, prior to alcohol use and prior to other drug use. The dose of naltrexone and dose of flumazenil selected are known to be safe and are in the order of 100 to 400 time lower than the safe dose.

The pilot studies also suggest that the medication allows patients to decrease their alcohol use and also their smoking and if we validate that this medication is effective in helping young volunteers to cease their alcohol and smoking patterns, then this will have enormous benefits in the management of unsocial behaviour in our society in the long term. There will also be a reduction in the requirement of hospital care, with the number of bed days saved in the short term, and also in the long term with reduced requirement for bed days in adult patients suffering from chronic liver failure, lung damage and a multitude of cancers caused by alcohol and tobacco.

The present study involves randomly recruiting 50 to 60 individuals under the age of 24 who have a history of excessive alcohol intake for a 4 week blinded crossover study. The study will involve intensive monitoring for the 4 week study with two weeks of placebo and 2 weeks of treatment.

These individuals will be recruited and will be considered suitable for the project providing they have an intention to address their alcohol, smoking and drug problems. It is expected that individuals will be recruited and managed via a Section-4 engagement and collaboration. The individuals will be further monitored for up to 12 weeks to measure whether benefits can be sustained on extended treatment.

### Recruitment Design

For the first 4 weeks in the cross over study, individuals will be randomised to receive the placebo or treatment in the first 2 weeks. In the second 2 weeks they will receive the opposite to the initial 2 weeks. Individuals will be informed that they may be given placebo or treatment at any time but will only be told which treatment they have received at the end of the 4 week treatment period.

If their addictive behaviour with alcohol and other substances was reduced in the time of treatment with the medicament they will be given the option to continue treatment for up to 12 weeks to measure sustainability of response and also measure adverse events.

### Patient Information

During the four week period the individuals will be given lozenges to dissolve in the mouth which is anticipated to help decrease the craving. The individuals will be randomised into one of two groups: to receive the composition comprising a mixture of naltrexone and flumazenil or placebo lozenges. The individual/patient and their GP will not be informed on which medication the patient received until the 4 week trial is completed. The results will be analysed at the end of 4 weeks and if the treatment helped the patient stop addictive substances then you will be invited to use the treatment for up to 3 months to assess the sustainability of treatment

The patients will report whether their craving for alcohol, cigarettes and other drugs is reduced in the time following taking the treatment or placebo, and also record use of alcohol, cigarettes and other drugs.

At the end of the four week trial, the patients in the placebo group and in the treatment group will be unblinded and be given the opportunity to now have the treatment available for a 3-month period. Those that take up the offer will then be monitored for the following 3-months during which time they will record whether their alcohol, smoking and drug use is reduced compared to their past history.

The treatment group will receive the composition in lozenge formulation comprising a mixture of 250 micrograms of naltrexone with 250 micrograms flumazenil. The lozenge will dissolve rapidly within the mouth. Alternatively, the composition may be formulated into a nasal spray comprising a mixture of 250 micrograms of naltrexone with 250 micrograms flumazenil. The nasal spray will provide a rapidly absorbed dose of naltrexone and flumazenil.

The placebo group will be given lozenges or nasal spray devoid of naltrexone or flumazenil.

It is anticipated that at the end of the initial four week trial, the group administered the composition of the present invention will have a significantly better outcome than those on the placebo treatment. That is, outcomes in the treatment group will include (i) a decrease in cravings, (ii) decrease in alcohol use, (iii) decrease in smoking, (iv) decrease in craving and use, and (v) decrease in drug craving and use.

Following the initial four week trial, the treatment will be made available to the individuals via sub-lingual delivery or alternatively as an intra-nasal delivery on a ready to use basis and on demand for a further 12 week (3 month period). In this regard, the individuals will be instructed to administer the treatment at the onset of the craving for alcohol, cigarettes, marijuana or other drugs. Since the doses of naltrexone and flumazenil are up to 100 time less than the dose known to be safe, the individuals will be encouraged to use up to 20 doses a day and in extreme cases of cravings, up to 40 doses a day.

It is anticipated that the 12-week study following the initial treatment with the composition of the present invention will help to define clearly whether the medicine is useful to patients in ceasing their alcohol, smoking and drug use over a 12-week period as a sustained cessation of these activities. It is expected that we will try to manage 5-10 patients at a time in groups, with a view to a full study of 60 patients.

### Parameters to Monitor Throughout Treatment

The following behavioural traits and habits will be monitored throughout the four week trial and during the following 12 weeks; alcohol use; marijuana use, smoking of cigarettes, amphetamine use, ecstasy use, opiate use, other drug use, gambling, benzodiazepine use, involvement in criminal activities, behavioural activity and mental health outcomes.

Most young alcohol using people in our community are affected by craving for a variety of substances such as cigarettes, alcohol, amphetamines, benzodiazepines and other substances.

Patients describe craving as being associated with craving induced anxiety that gradually rises to a level where drug use becomes inevitable. In some cases drug seeking behaviour is automatic with little anxiety but as counselling demands changes to cease drug seeking awareness of craving anxiety often increases. The treatment aims to combine teaching to increase awareness of drug seeking behaviour and provide early treatment of rises in craving anxiety so that drug use is decreased.

The following are expected to arise from the administration of the medicament of the present invention: decrease in background craving, decrease in craving anxiety 15 and 30 minutes post treatment, decrease in substance use in response to craving, decrease rate in each addictive substance or behaviour and sustainability of responses over 12 weeks follow up treatment in responders to pharmacotherapy.

### Example 6

The aim of a further trial is to recruit 40 patients into a randomised control trial with 20 treated with placebo and 20 treated with treatment implants. The results from the 20 individuals allocated to the placebo group would be monitored for 8 weeks before unblinding and would then be offered treatment implants as a follow up treatment in an open non blinded crossover for another 8 weeks.

### Recruitment

Patients aged from 14 to 24 years will be recruited from various hospitals or newspaper advertisements. Potential individuals having a history of alcohol abuse will be invited to join the trial. History of alcohol abuse is defined as drinking heavily for at least twice per week. A willingness to attempt abstinence from alcohol and achieve wellness will have to be displayed to be involved in the randomised controlled trial. Individuals with psychiatric disorders, epilepsy controlled with benzodiazepines, tricyclics antidepressants users or Cocaine users would be excluded from the trial.

### Implant-Treatment Group Versus Control Group

The treatment group will receive implants that deliver 10 to 18 mg of naltrexone per day and 1-3 mg of flumazenil a day. The control group receive implants devoid of naltrexone and flumazenil.

### Patient Information and Monitoring Parameters

Information will be collected from patients as set out above in Example 5. Similar parameters will be measured as set out in Example 5.

It is expected that the use of flumazenil and naltrexone delivered with implants modifies the GABA response and opioid response with a continuous blood level not easily obtained by other means.

It is expected that patients treated with the treatment implant will experience a decrease in background craving, decrease in craving anxiety 15 and 30 minutes post treatment, decrease in substance use in response to craving, decrease rate in each addictive substance or behaviour and sustainability of responses over 12 weeks follow up treatment in responders to pharmacotherapy.

### Example 7

A female indigenous patient who smoked around 50 cigarettes a day was administered a dose of bupropion (250 micrograms) in combination with 250 micrograms of flumazenil and 250 micrograms of naltrexone via nasal delivery. The bupropion appeared to maximise the effectiveness of the flumazenil/naltrexone combination. Within minutes the cravings associated with smoking ceased and the patient has not smoked a cigarette in over 120 days.

Every time the patient had a craving for a cigarette, she administered one spray of the composition, which delivered 250 μg bupropion, 250 μg naltrexone and 250 μg flumazenil per dose. As the cravings became less frequent, the patient did not require treatment via the nasal spray combination of bupropion/flumazenil/naltrexone as often and as such, the patient now only requires treatment three doses per day after 120 days of treatment.

It is anticipated that this combination of active agents delivered as pulsatile doses via a nasal spray will assist with symptomatic relief of “hangovers” associated with alcohol consumption and benzodiazepine use. It is suggested that the nasal spray of bupropion/flumazenil/naltrexone is administered before sleep. Administration may be in the form of one or two nasal sprays, wherein each “spray” administers bupropion (250 μg), flumazenil (250 μg) and naltrexone (250 μg) per dose.

The combination of bupropion/flumazenil/naltrexone is also suggested for the treatment of food cravings (i.e. to reduce food intake), depression, anxiety, gambling addiction, and other addictions associated with impulsive behaviour, where a dose of 250 μg each of bupropion, flumazenil and naltrexone is administered every 15 minutes until the symptoms are alleviated, or one spray every time an episode of craving, depression or anxiety is experienced by the patient.

### Example 8

Patients presenting with Attention Deficit Disorder (ADD) or Attention Deficit Hyperactivity Disorder (ADHD) were treated with a combination of flumazenil (250 micrograms) and naltrexone (250 micrograms) via nasal spray administration. Each single spray delivered a dose of 250 μg each of flumazenil and naltrexone to the patient.

Immediately following treatment (i.e. within minutes of receiving the treatment), we observed a reduction in symptoms associated with ADD and ADHD. The patients' thoughts became clearer, films could be watched, books could be read and the shaking of the legs stopped. This symptom relief for ADD/ADHD with the combined administration of naltrexone and flumazenil in a spray delivery is a new observation and was totally unexpected.

### Example 9

We observed a cessation in cravings for cigarettes when patients were treated with a composition comprising a dose of 15 micrograms clonidine, 250 micrograms naltrexone and 250 micrograms of flumazenil in a nasal spray, administered per dose per single spray.

1. A composition comprising a micro-dose of naltrexone in combination with a micro-dose of flumazenil.

2. A composition according to claim 1 wherein the micro-dose of naltrexone ranges from about 10 microgram to about 2000 micrograms and the micro-dose of flumazenil ranges from about 10 microgram to about 2000 micrograms.

3. A composition comprising a micro-dose of naloxone in combination with a micro-dose of flumazenil.

4. A composition comprising a micro-dose of nalmephene in combination with a micro-dose of flumazenil.

5. A composition according to claim 3 or claim 4 wherein the micro-dose of naloxone or nalmephene ranges from about 10 microgram to about 2000 micrograms and the micro-dose of flumazenil ranges from about 10 microgram to about 2000 micrograms.

6. A composition according to any one of claims 1 to 4 comprising 250 micrograms of naltrexone, or naloxone or nalmephene and 250 micrograms of flumazenil.

7. A composition comprising a micro-dose of bupropion in combination with a micro-dose of naltrexone and a micro-dose of flumazenil.

8. A composition comprising a micro-dose of clonidine in combination with a micro-dose of naltrexone and a micro-dose of flumazenil.

9. A composition according to claim 7 wherein the micro-dose of bupropion ranges from about 10 micrograms to about 2000 micrograms, the micro-dose of naltrexone ranges from about 10 microgram to about 2000 micrograms and the micro-dose of flumazenil ranges from about 10 microgram to about 2000 micrograms.

10. A composition according to claim 8 wherein the micro-dose of clonidine ranges from about 10 micrograms to about 150 micrograms, the micro-dose of naltrexone ranges from about 10 microgram to about 2000 micrograms and the micro-dose of flumazenil ranges from about 10 microgram to about 2000 micrograms.

11. A composition according to any one of claims 1 to 10 wherein the composition is delivered by rapid delivery or bolus delivery systems.

12. A composition according to claim 11 wherein the delivery is selected from the group consisting of: sublingual, nasal spray, intra-nasal delivery, rectal, intra-pulmonary, or subcutaneous injection.

13. A composition according to any one of claims 1 to 12 when used in the treatment of craving or anxiety associated with dependency.

14. The composition according to claim 13 for the treatment of craving associated with the endogenous opioid system, including opioids.

15. A method of treating an individual suffering from craving or anxiety associated with dependency comprising administering a composition comprising a micro-dose of naltrexone in combination with a micro-dose of flumazenil.

16. A method of treating an individual suffering from craving or anxiety associated with dependency comprising administering a composition comprising a micro-dose of bupropion in combination with a micro-dose of naltrexone and a micro-dose of flumazenil.

17. A method of treating an individual suffering from craving or anxiety associated with dependency comprising administering a composition comprising a micro-dose of clonidine in combination with a micro-dose of naltrexone and a micro-dose of flumazenil.

18. The method according to claim 15 wherein the micro-dose of naltrexone ranges from about 10 microgram to about 2000 micrograms and the micro-dose of flumazenil ranges from about 10 microgram to about 2000 micrograms.

19. The method according to claim 16 wherein the micro-dose of bupropion ranges from about 10 micrograms to about 2000 mg, the micro-dose of naltrexone ranges from about 10 microgram to about 2000 micrograms and the micro-dose of flumazenil ranges from about 10 microgram to about 2000 micrograms.

20. The method according to claim 17 wherein the micro-dose of clonidine ranges from about 10 micrograms to about 150 mg, the micro-dose of naltrexone ranges from about 10 microgram to about 2000 micrograms and the micro-dose of flumazenil ranges from about 10 microgram to about 2000 micrograms.

21. The method according to claim 17 or 18 wherein the micro-dose of naltrexone is 250 micrograms and the micro-dose of flumazenil is 250 micrograms.

22. The method according to any one of claims 15 to 21 wherein the craving is a craving for cigarettes, marijuana, alcohol, cocaine, heroin, amphetamines, or other drugs.

23. Use of a composition comprising a micro-dose of naltrexone in combination with a micro-dose of flumazenil in the preparation of a medicament for the treatment of opioid dependency.

24. Use of a composition comprising a micro-dose of bupropion in combination with a micro-does of naltrexone and a micro-dose of flumazenil in the preparation of a medicament for the treatment of opioid dependency.

25. Use of a composition comprising a micro-dose of clonidine in combination with a micro-does of naltrexone and a micro-dose of flumazenil in the preparation of a medicament for the treatment of opioid dependency.

26. Use of a composition according to claim 22 wherein the dependency is craving for cigarettes, marijuana, alcohol, cocaine, heroin, amphetamines, or other drugs.
